(19) United States
(12) Patent Application Publication
(10) Pub. No.: US 2021/0031054 A1
(43) Pub. Date: Feb. 4, 2021

(54) DECISION SUPPORT TOOL FOR ADAPTIVE RADIOTHERAPY IN CT/LINAC CONSOLE

(71) Applicant: KONINKLIJKE PHILIPS N.V., EINDHOVEN (NL)

(72) Inventors: Vaitheeswaran RANGANATHAN, BANGALORE (IN); Bojarajan PERUMAL, BANGALORE (IN); Rohinee NUGGEHALLI ASHWINEE KUMAR, BANGALORE (IN); Saravanan KARUNANITHI, CHENNAI (IN)

(21) Appl. No.: 17/040,920

(22) PCT Filed: Mar. 18, 2019
(86) PCT No.: PCT/EP2019/056641

§ 371 Date: Sep. 23, 2020

Related U.S. Application Data
(60) Provisional application No. 62/647,963, filed on Mar. 26, 2018.

(51) Int. Cl. A61N 5/10; G16H 20/40; G16H 30/40

(52) U.S. Cl. CPC A61N 5/1039; A61N 5/1077; A61B 6/032; G16H 30/40; A61N 2005/1074; G16H 20/40

(57) ABSTRACT

A radiation therapy delivery device console (50) controls a radiation therapy delivery device (36) and an imaging device (40, 42), and further performs adaptive radiotherapy (ART) recommendation as follows. The imaging device is controlled to acquire a current image (44) of a patient. At least one perturbation of the current image is determined compared with a radiation therapy planning image (1) from which a radiation therapy plan (22) for the patient has been generated. An ART recommendation score is computed, indicating whether ART should be performed, based on the determined at least one perturbation. A recommendation is displayed as to whether ART should be performed based on the computed ART recommendation score, or an alarm is displayed conditional upon the computed ART recommendation score satisfying an ART recommendation criterion.

### FIELD

The following relates generally to the radiation therapy arts, radiation therapy planning arts, adaptive radiotherapy arts, and related arts.

### BACKGROUND

Radiation therapy is a common treatment for certain types of cancers. In general, the goal is to deliver a prescription dose of radiation to a tumor or other malignant tissue while minimizing radiation exposure to surrounding healthy tissue, and especially to so-called critical tissues or organs at risk (OARs). A linear accelerator (linac) or other radiation therapy delivery device is employed to deliver the therapeutic radiation (e.g. energetic electrons, protons, or x-rays). A linac includes a rotating gantry enabling tomographic delivery of therapeutic radiation at various angles around the patient, and may operate in a step-and-shoot or continuous deliver mode depending upon the linac design and the prescribed radiation therapy protocol. The radiation beam is modulated during the tomographic delivery, hence the technique is commonly referred to as intensity modulated radiation therapy (IMRT). Other radiation therapy delivery techniques may be employed, such as three-dimensional conformal radiation therapy (3D-CRT), intensity-modulated arc therapy (IMAT), and volumetric modulated arc therapy (VMAT), or so forth. Likewise, other radiation therapy delivery devices may be employed beside a linac, such as devices which deliver multiple radiation beams simultaneously. In many radiation therapy protocols, fractionated radiation therapy delivery is employed, in which the total radiation dosage is delivered over multiple sessions (i.e. “fractions”) that may be separated by days or longer. Fractionated radiation therapy has been found to increase efficacy and reduce long-term damage to OARs.

To deliver effective radiation therapy, a radiation treatment plan is developed for the individual patient. This entails acquiring a detailed planning image of the relevant anatomy of the individual patient by a radiological imaging modality such as transmission computed tomography (CT) or magnetic resonance imaging (MRI), possibly augmented by functional information on the tumor or other malignant tissue provided by an emission imaging modality such as positron emission tomography (PET) or single photon emission computed tomography (SPECT). The planning image is contoured to delineate the tumor(s) and neighboring OARs using manual, automated, or semi-automated contouring (usually with any automatically generated contours being reviewed and adjusted as appropriate by an oncologist or other qualified medical professional). The oncologist develops objectives for the therapy, such as a prescription dose to be delivered to each tumor and maximum dosages for OARs or the like. A Treatment Planning System (TPS) is then applied to generate an individualized radiation treatment plan for the patient. This is usually done by a specialist, sometimes referred to as a radiation physicist, using an “inverse” process in which initial settings or parameters of the radiation delivery device (e.g. linac) are set and the resulting radiation fluence or dose distribution is simulated by the TPS and compared with the plan objectives established by the oncologist, and then the settings/parameters are adjusted and the fluence or dose distribution recomputed in an iterative fashion until optimized settings/parameters are obtained that substantially satisfy the objectives. During this process, the number of fractions (in the case of a fractionated radiation therapy protocol) may also be varied to ascertain and optimize the impact of the number of fractions. In spite of having a large number of settings/parameters and adjusting the fractions, in practice the final radiation treatment plan may not fully satisfy all objectives. The oncologist reviews the proposed radiation treatment plan and may approve it, or alternatively may disapprove the plan and identify area(s) where improvement is required. In case of disapproval, the radiation physicist performs further optimization using the TPS and submits an updated treatment plan to the oncologist, until an approved plan is reached.

When a radiation therapy session (i.e. fraction) is to be performed, the patient arrives at the treatment location and is positioned on a patient support. This positioning is critical since the tumor(s) and OARs should be in substantially the same position as during the acquisition of the CT or MRI planning image from which the radiation therapy plan was developed. To assist in positioning, many linacs or other radiation delivery devices include a built-in CT imaging (sub-)system to acquire an image of the patient positioned on the therapy patient support just prior to commencement of the radiation therapy delivery. This is often a cone-beam CT (CBCT) imaging system. This CBCT image is used to assist in patient positioning.

Adaptive radiotherapy (ART) is a variant approach, in which the radiation therapy plan for a fraction may be adjusted just prior to commencement of radiation delivery based on the CBCT image. ART is designed to account for the fact that patient anatomy may change over time. For example, a bladder may be more or less full during one session versus another, organs can shift within the body, the patient may gain or lose weight, the malignant tumor(s) may shrink or grow, and et cetera. These changes are accommodated by adjusting the radiation therapy plan itself. To this end, the image newly acquired by the CBCT at the linac is sent to the TPS, where a radiation physicist contours the image and simulates the fluence or dose distribution for the newly acquired image using the current radiation treatment plan settings/parameters for the fraction. Based on this simulation, a determination is made as to whether ART should be performed to update the radiation treatment plan. If so, then the treatment plan is updated by performing further optimization now using the newly acquired image. If it is determined that no ART is needed, then this decision is communicated back to the radiation therapy delivery laboratory where radiation delivery is performed using the existing plan.

The following discloses certain improvements.

### SUMMARY

In some embodiments disclosed herein, a non-transitory storage medium stores instructions readable and executable by a console including a display, at least one user input device, and an electronic processor to perform a method comprising: determining at least one perturbation of a current image compared with a radiation therapy planning image used to generate a radiation therapy plan; computing an adaptive radiotherapy recommendation score indicating whether adaptive radiotherapy should be performed by operations including applying a radiation therapy plan-specific perturbation model that is specific to the radiation therapy plan and is functionally dependent on the determined at least one perturbation; and displaying, on the display of the console, one of (i) a recommendation as to whether adaptive radiotherapy should be performed based on the computed adaptive radiotherapy recommendation score and (ii) an alarm conditional upon the computed adaptive radiotherapy recommendation score satisfying an ART recommendation criterion.

In some embodiments disclosed herein, a console comprises a display, at least one user input device, an electronic processor, and a non-transitory storage medium storing instructions readable and executable by the electronic processor to control a radiation therapy delivery device operatively connected with the console. The instructions are further readable and executable by the electronic processor to perform a method including: receiving a current image of a patient; determining at least one perturbation of the current image compared with a radiation therapy planning image from which a radiation therapy plan for the patient has been generated; computing an adaptive radiotherapy recommendation score indicating whether adaptive radiotherapy should be performed based on the determined at least one perturbation; and displaying, on the display, one of (i) a recommendation as to whether adaptive radiotherapy should be performed based on the computed adaptive radiotherapy recommendation score and (ii) an alarm conditional upon the computed adaptive radiotherapy recommendation score satisfying an ART recommendation criterion.

In some embodiments disclosed herein, a radiation therapy delivery system comprises: a radiation therapy delivery device configured to deliver therapeutic radiation to a patient disposed on a patient support; an imaging device configured to image the patient disposed on the patient support of the radiation therapy delivery device; and a console as set forth in the immediately preceding paragraph, which is operatively connected to control the radiation therapy delivery device and to control the imaging device. In some embodiments, the radiation therapy delivery device comprises a linear accelerator (linac) and the imaging device comprises a computed tomography (CT) scanner.

In some embodiments disclosed herein, an adaptive radiotherapy recommendation method comprises: determining at least one perturbation of a current image of a patient compared with a radiation therapy planning image of the patient from which a radiation therapy plan for the patient has been generated; computing an adaptive radiotherapy recommendation score indicating whether adaptive radiotherapy should be performed based on the determined at least one perturbation and without simulating a dose distribution in the patient as represented by the current image; and controlling a display to present one of (i) a recommendation as to whether adaptive radiotherapy should be performed based on the computed adaptive radiotherapy recommendation score and (ii) an alarm conditional upon the computed adaptive radiotherapy recommendation score satisfying an ART recommendation criterion. The adaptive radiotherapy recommendation method is suitably performed by an electronic processor.

One advantage resides in providing for reduced delay in determining whether adaptive radiotherapy (ART) should be performed.

Another advantage resides in providing a principled basis for deciding whether ART should be performed.

Another advantage resides in reduced workload for the radiation physicist or other operator of the Treatment Planning System (TPS).

Another advantage resides in reduced computational workload on the TPS.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The invention may take form in various components and arrangements of components, and in various steps and arrangements of steps. The drawings are only for purposes of illustrating the preferred embodiments and are not to be construed as limiting the invention.

FIG. 1 diagrammatically illustrates principle systems and devices involved in planning and delivery of radiation therapy including ART recommendation as disclosed herein.

FIG. 2 diagrammatically illustrates radiation therapy planning and delivery processing suitably performed by the setup of FIG. 1.

FIGS. 3-5 diagrammatically illustrate simulation results as described herein.

### DETAILED DESCRIPTION

In Adaptive Radiotherapy (ART), the treatment plan is often adapted for changing anatomy of the patient. Adapting the plan involves acquiring a new CBCT image of the patient, recontouring of tumor volume and organs in the newly acquired image, plan quality assessment and optimizing the beam parameters for the changed contours. Not performing ART can lead to potentially partially or completely missing target volume and/or excessive dose delivered to surrounding normal tissues, e.g. organs at risk (OARs). On the other hand, ART consumes substantial computational resources, as well as valuable time of the radiation physicist or other highly skilled TPS operator. ART also delays delivery of the radiation fraction, which is disturbing for the patient and may cause a backlog for the radiation therapy delivery laboratory.

In existing approaches, it is not known whether ART is appropriate until the plan quality assessment or evaluation is done for the changed anatomy. Typically it takes about 30 to 45 minutes to complete the plan quality assessment to decide the need for ART (excluding the time taken for acquiring the CBCT images). The patient typically cannot stay in the same position for such a long time. Basically the following processes are involved post image acquisition to complete the plan evaluation: 1. Transferring of the newly acquired CBCT image set to the treatment planning system (TPS); 2. Performing re-contouring and/or deformable image registration (DIR); 3. Performing plan evaluation for the changed contours; and 4. Making the decision as to whether ART should be performed.

Some drawbacks in the foregoing approach are the need for transferring the image data to the TPS, the computational load of performing the re-contouring and simulation of the fluence or dose distribution (the latter often performed ab initio in the TPS), the additional workload on the radiation physicist, and potential delays introduced by the need to coordinate operations at two different locations (the TPS and the radiation therapy delivery laboratory). In many clinics the availability of TPS for evaluating the need for ART is limited, which adds to the delay. In view of these difficulties, ART may be avoided entirely in some situations, which can lead to delivering a sub-optimal treatment.

In embodiments disclosed herein, the decision as to whether ART should be performed is made with the patient lying on the couch preparatory to radiation therapy delivery, and takes as short as a few minutes (excluding the time taken for acquiring the CBCT images). In some embodiments, the decision as to whether ART should be performed is made locally at the CT/Linac console, without consulting the TPS. If it is decided, at the CT/Linac console, that ART is not needed, then the radiation therapy delivery can commence immediately using the existing radiation therapy plan. On the other hand, if it is decided that ART should be performed, then the CBCT image is transferred to the TPS where ART is performed per usual procedure.

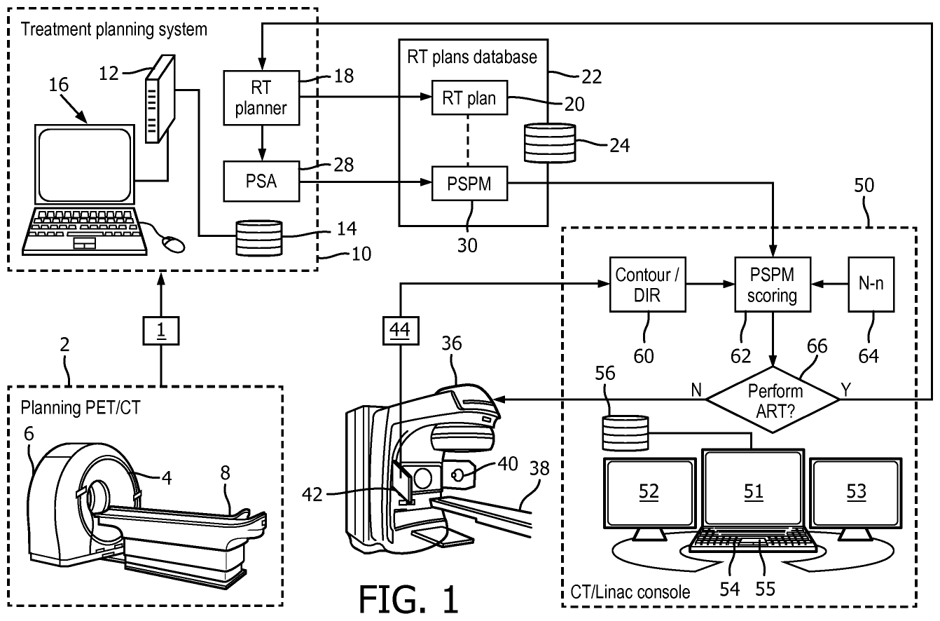

With reference to FIG. 1, principle systems and devices involved in planning and delivery of radiation therapy are diagrammatically shown, including a CT/Linac-based ART recommender as disclosed herein. To begin the process, a radiation therapy planning image 1 to be used in generating a radiation therapy plan is acquired by a suitable medical imaging device, such as an illustrative PET/CT scanner 2 having a computed tomography (CT) gantry 4 and a positron emission tomography (PET) gantry 6 coupled by a common patient support 8 via which a prone or supine patient can be moved into either gantry 4, 6 for CT and/or PET imaging. Typically, CT images show detailed anatomy and are used for the radiation therapy planning process, while PET images provide functional information and show tumors as brighter “hot spots”. Although PET advantageously provides such functional information, in some embodiments the PET imaging may be omitted, e.g. the imaging device may be a standalone CT scanner. Moreover, the illustrative PET/CT scanner 2 is merely an illustrative example, and it will be appreciated that other imaging modalities may additionally or alternately be employed to acquire the radiation therapy planning image 1, such as a magnetic resonance imaging (MRI) scanner.

The radiation therapy planning image 1 serves as input to a Treatment Planning System (TPS) 10 which in the illustrative embodiment comprises an illustrative server computer 12 or other electronic data processing device with substantial computing capacity. The electronic processor 12 reads and executes instructions stored on a non-transitory storage medium 14 to perform complex radiation fluence and/or dose distribution calculations and dose optimizations. The TPS 10 includes or is accessed by a user interface 16 with a display and at least one user input device (e.g., mouse, keyboard). More generally, the TPS 10 may be implemented using alternative computing hardware, such as a cloud-based computing resource or other distributed computing system, and/or may employ alternative user interfacing arrangements, e.g. the illustrative user interface 16 may be replaced by log-in access by generic computers, tablet computers, or the like to provide distributed TPS access and operation. The TPS 10 performs dose optimizations by an inverse planning procedure in which parameters of the radiation therapy device to be employed are selected and the resulting fluence and/or dose distribution in the patient achieved by those parameters is simulated, and a suitable iterative optimization is applied to adjust the parameters to bring the simulated dose distribution into compliance (to the extent practically achievable) with a set of goals or objectives defined by the patient's physician or oncologist. By way of non-limiting illustration, the objectives may include dose specifications defining a therapeutically effective dosing of the tumor under treatment, and maximum permissible dose specifications for one or more organs at risk (OARs) located close to the tumor and hence necessarily receiving some (undesired) radiation dose. The dose optimization is computationally complex and commonly involves tens of minutes to hours of computing time, and is managed by a radiation physicist or other specialized medical professional, possibly in consultation with the patient's physician or oncologist. In many radiation therapy regimens, the dose delivery is administered in several successive sessions, called “fractions”, delivered on subsequent days possibly extending over a period of weeks or months—such a radiation therapy regimen is referred to as fractionated radiation therapy. Without loss of generality, the number of fractions is denoted herein as N, and the number of fractions that have been completed to the present time is denoted as n. Thus, the number of remaining fractions at the present time is N−n.

As diagrammatically illustrated in FIG. 1, the radiation treatment planning is performed by the electronic processor 12 (e.g. server computer) executing instructions stored on the non-transitory storage medium 14 to implement a radiation treatment planner 18 that performs the dose distribution simulations and the dose optimization process. By way of non-limiting illustrative example, the radiation treatment planner 18 could be embodied by the Pinnacle³ Treatment Planning tool available from Koninklijke Philips N.V. The radiation therapy plan 20 generated by the dose optimization performed by the radiation treatment planner 18 and approved by the patient's physician or oncologist is stored in a radiation therapy plans database 22 stored on a non-transitory storage 24 for later retrieval when a fraction of the radiation therapy regimen is to be performed.

In embodiments disclosed herein, the TPS 10 further performs a plan sensitivity analysis (PSA) 28 implemented by the electronic processor 12 (e.g. server computer) executing instructions stored on the non-transitory storage medium 14 in order to generate a plan-specific perturbation model (PSPM) 30. After the radiation therapy planner 18 under management of the radiation physicist generates the radiation therapy plan based on the radiation therapy planning image 1 (and approved by the physician or oncologist), and before commencing the first fraction of treatment, the plan sensitivity analysis (PSA) 28 is performed. In PSA, various deformation scenarios suggested by the clinician (or integrally stored in the PSA 28) are simulated in the radiation therapy planning image 1 itself and the impact on the radiation treatment plan quality is stored. A set of valid perturbations may be defined per anatomic site and can be kept in a library (not shown) stored on the non-transitory storage medium 14, which can be input to the PSA 28. Optionally, a weightage factor per perturbation may be specified so that important deformation scenarios are given more importance in the generated PSPM 30. Such weighting may provide a mechanism for assigning appropriate significance to various perturbations dependent upon factors such as the clinical case (e.g. a perturbation in the bladder may be more important for radiation therapy of the prostate as compared with radiation therapy of another organ with less proximity to the bladder), patient age, patient-specific priorities (e.g. different patients may be more or less concerned about certain potential adverse effects of radiation therapy), and/or so forth.

For example, some perturbations that may be so analyzed include (by way of non-limiting illustrative example): expansion or contraction of the urinary bladder; expansion or contraction of the tumor under treatment; patient weight gain or loss; normal shifting of various internal organs; various combinations thereof; and so forth. Each of these may be further subdivided, e.g. shrinkage or growth of the tumor in the anterior-posterior direction may be one analyzed perturbation; shrinkage or growth of the tumor in the superior-inferior direction may be another analyzed perturbation; and shrinkage or growth of the tumor in the lateral direction may be another analyzed perturbation; and similar anisotropies in other possible perturbations. The analyzed perturbation may also be in some oblique direction is anatomically appropriate. Urinary bladder perturbation may be similarly modeled; alternatively, if it is known that the urinary bladder grows or shrinks in an approximately isotropic fashion (e.g. due to fluid expanding the bladder uniformly in all directions) then the modeled bladder expansion/shrinkage may be a single perturbation. While physical shifting, growth, or shrinkage are typical perturbations appropriate for the PSA to analyze, other types of perturbations are also contemplated, e.g. hardening or calcification of tissue could be another analyzed perturbation. Moreover, if certain perturbations are expected to be correlated then a combined perturbation may be defined, e.g. shrinkage of the tumor may produce a consequent expansion of a contacting OAR so that the combined perturbation is the shrinkage of the tumor and correlated expansion of the OAR. More generally, a combined perturbation may be defined to represent any situation in which a combination of individual perturbations may produce a synergistic effect that is not well-described by considering each individual perturbation in isolation. The impact of a perturbation of a given amount may be quantified in various ways, for example by computing a percent change in the value of the composite objective function used in the dose optimization (the composite combines the individual objectives for the tumor and various OARs) produced by the perturbation.

After simulating sufficient number of scenarios (i.e. perturbations of various amounts), the PSA 28 generates the PSPM 30 which relates a specific perturbation in the anatomy (e.g., growth of the urinary bladder by a specific amount; as mentioned previously, the specific perturbation could be a combined perturbation, i.e. a combination of two or more individual perturbations in order to capture correlative effects) to the corresponding impact on the plan quality. In one approach, a pre-defined anatomy-specific template of acceptable level of impact on the plan quality is used to generate an adaptive radiotherapy recommendation score. The adaptive radiotherapy recommendation score may, for example, be expressed as a risk, i.e. the amount of risk involved in delivering a certain plan on a certain fraction of the treatment for the current anatomy. More generally, the adaptive radiotherapy recommendation score indicates whether adaptive radiotherapy should be performed, and is computed by operations including applying the radiation therapy plan-specific perturbation model 30, which is specific to the radiation therapy plan 20 and is functionally dependent on one or more perturbations as discussed previously. The PSPM 30 is suitably stored with the radiation therapy plan 20 for retrieval when a fraction of the radiation therapy regimen is to be performed.

With continuing reference to FIG. 1, the foregoing processing generating the patient-specific radiation therapy plan 20 and the corresponding PSPM 30 is performed “offline”, that is, before delivery of therapeutic radiation during the first fraction of the fractionated radiation therapy. Optionally, the PSA 28 performed to generate the PSPM 30 can be automated such that right after creation of the radiation treatment plan 20, the TPS 10 automatically runs the PSA 28 in the background and sends the PSPM 30 to the storage 22 and optionally to the linac console 50 (to be described) as well. It is also contemplated to refine the PSPM 30 using additional processes, for example using deep learning techniques to modify the model based on prior information such as clinical outcomes mined from previous patient cases or so forth.

The radiation therapy (or each radiation therapy fraction in the case of a fractionated radiation therapy regimen) is performed by a radiation therapy delivery device 36. In the illustrative example, the radiation therapy delivery device 36 is a linear accelerator (linac) that generates therapeutic radiation by accelerating electrons to high energy (typically above 1 MeV). The therapeutic radiation may be the high energy electrons, or may be X-rays generated by directing the high energy electron beam to an X-ray generating target such as a tungsten target. In other embodiments, the radiation therapy delivery device may be some other type of particle accelerator, e.g. generating therapeutic radiation in the form of a proton beam, as another non-limiting illustrative example. Preparatory to performing the radiation therapy fraction, the patient is positioned on a patient support 38. This is done in a precise manner, using fiduciary markers and/or anatomical markers to ensure alignment of the patient anatomy with its positioning during acquisition of the radiation therapy planning image 1, and may entail applying appropriate restraints to hold the patient in the appropriate position.

To assist in positioning the patient and to assess whether major changes in patient anatomy have occurred since acquisition of the radiation therapy planning image 1, an imaging device 40, 42 is configured to image the patient disposed on the patient support 38 of the radiation therapy delivery device 36. This imaging device 40, 42 is different from the imaging device 2 used to acquire the radiation therapy planning image 1. In the illustrative embodiment, the imaging device 40, 42 is a cone beam computed tomography (CBCT) imaging component 40, 42 of the radiation therapy delivery device 36, and includes a cone beam X-ray source 40 and an x-ray detector array or panel 42 positioned across the patient support 38 from the X-ray source 40. A CBCT imaging component has certain advantages—it provides a current image 44 which has transmission CT contrast making it directly comparable with the radiation therapy planning image 1 (at least in the illustrative example in which the planning image 1 is also a CT image). However, more generally the imaging device associated with the radiation therapy delivery device 36 may be any medical imaging device capable of acquiring the current image 44 of the patient that can be compared with the radiation therapy planning image 1.

As another example, the imaging device associated with the radiation therapy delivery device may be a magnetic resonance imaging (MRI) device, either standalone or integrated with the linac to form an MR-LINAC in which the MR imaging device is part of the LINAC itself (analogous to the illustrative CT-Linac 36, 40, 42 illustrated in FIG. 1). Since MRI does not transmit ionizing radiation into (or through) the subject, it can be used on daily basis to acquire a current image prior to each radiation therapy session without concern about increasing the cumulative radiation dose to the patient. The availability of MR images can be leveraged, and the ART recommendation calculations performed in the background so as to alert the linac operator if something is wrong. As yet another example, the imaging device associated with the radiation therapy delivery device may be an MR/CT imaging device providing both MR and CT imaging modalities, in which the MR and CT images may be aligned or correlated. MR/CT advantageously provides different and sometimes complementary contrast mechanisms that can elucidate more information than either MR or CT alone. The illustrative imaging device 40, 42 associated with the radiation therapy delivery device 36 is a component of the radiation therapy delivery device 36, e.g. mounted to the housing of the illustrative linac 36. However, this is not required—in another embodiment, the imaging device associated with the radiation therapy delivery device may be a portable imaging device on a wheeled support, which is rolled over to and aligned with the patient support 38 to acquire the current image 44.

The radiation therapy delivery device 36 and the imaging device 40, 42 are controlled by a console 50 which includes or has operative access to a display (in the illustrative example, three displays 51, 52, 53 to provide display area sufficient to display substantial information to be considered during the delivery of therapeutic radiation; more generally, one, two, three, or more displays may be provided) and one or more user input devices (e.g. an illustrative keyboard 54 and trackpad 55 or other pointing device, optionally one or more such user input pointing devices may be implemented by making one or more of the displays 51, 52, 53 a touch-sensitive display). The console 50 includes an electronic processor (e.g. microprocessor, microcontroller, et cetera) that reads and executes instructions stored on a non-transitory storage medium 56 to control the radiation therapy delivery device 36 which is operatively connected with the console 50, and to control the imaging device 40, 42 which is operatively connected with the console 50, and to perform an adaptive radiotherapy (ART) recommendation method as disclosed herein. The console 50 is illustrated as being disposed proximate to the radiation therapy delivery device 36; however, it will be understood that this proximity can be implementation-dependent, and that moreover some components of the console 50 may be located remotely. For example, depending upon radiation exposure control practices, the console 50 may be located in a different room from the radiation therapy delivery device 36 so as to limit the potential for stray radiation exposure to workers. The electronic processor and non-transitory storage medium 56 may be located remotely (e.g. implemented at a central hospital server). The console 50 may also have a “remote app” component, e.g. oncologists associated with the radiation therapy facility may be provided with cellphone, tablet, and/or desktop computer applications (“apps”) that provide for remote review of radiation therapy sessions, the current image 44, and/or so forth.

Conventionally, ART recommendation entails simulating the dose distribution for the current image 44 and the parameters of the radiation therapy delivery device 36 as given in the radiation therapy plan 20. Such simulation utilizes substantial computing resources and may require management by the radiation physicist or other medical person with specialized training. As such, it may not be practical to provide a dose distribution simulator at the linac console, and in some embodiments the disclosed linac console 50 follows this convention insofar as the linac console 50 does not include dose distribution simulation capability, that is, the non-transitory storage medium 56 does not store instructions readable and executable by the console 50 to perform dose distribution simulation for the current image 44. Conventionally, this deficiency of the linac console is handled by making the decision as to whether ART should be performed at the TPS 10. Thus, conventionally the current image 44 would be transmitted to the TPS 10, and the radiation physicist would perform the deformable image registration (DIR) of the current image with the planning image 1 and would perform contouring on the current image followed by dose distribution simulation for the current image, and then assess whether ART is advisable on the basis of the simulated dose distribution for the current image. If the decision is to not perform ART, then this decision would be communicated back to the operators at the linac console 50 who can then continue with delivery of therapeutic radiation in accord with the existing radiation treatment plan 20. Unfortunately even though ART is not performed the decision process itself can take 30 minutes or longer, introducing a substantial delay into the radiation therapy session. Consequently, the linac operators may elect to skip the ART review, possibly thereby missing out on benefits that ART could provide.

In embodiments disclosed herein, the plan-specific perturbation model (PSPM) 30 is used to generate an ART recommendation at the linac console 50, without the need to perform any dose distribution simulation and without the need to consult with the TPS 10. The ART recommendation can be generated in a matter of seconds or at most minutes. If the decision is to not perform ART, then delivery of therapeutic radiation can commence immediately, with a delay of only a few seconds or minutes to generate the ART recommendation based on the PSPM 30. On the other hand, if ART is recommended, then the current image 44 is transmitted to the TPS 10 and the updated adapted radiation treatment plan is received back from the TPS 10 and the therapeutic radiation is delivered in accord with that adapted plan.

To perform the ART recommendation method at the linac console 50, the current image 44 is acquired as usual, prior to commencement of delivery of the therapeutic radiation by the linac 36. Additionally, the radiation therapy planning image 1 is retrieved from the database 22. Deformable image registration (DIR) and feature contouring processing 60 is performed at the linac console 50 to spatially register the current image 44 with the planning image 1 and to define contours of the tumor and OARs (and/or other features of interest) in the current image 44. Note that such feature contouring was already done on the planning image 1 by the radiation physicist and/or oncologist or other medical professional as part of the radiation treatment planning 18, and the contours are preferably stored in the database 22 with the planning image 1 as part of (or as additional data associated with) the stored radiation therapy plan 20. Thus, only the current image 44 needs to be contoured. In an operation 62, an adaptive radiotherapy recommendation score is computed, which indicates whether adaptive radiotherapy should be performed. The operation 62 includes determining at least one perturbation of the current image 44 compared with the radiation therapy planning image 1 (which, again, was used to generate a radiation therapy plan 20), and applying the radiation therapy plan-specific perturbation model (PSPM) 30 that is specific to the radiation therapy plan 20 for the patient. Each perturbation is determined as a change in the at least one feature contoured in the current image compared with the at least one feature contoured in the spatially registered radiation therapy planning image.

As previously discussed, the PSPM 30 is functionally dependent on the determined at least one perturbation. In the case of a fractionated radiation therapy regimen, the PSPM 30 may be further functionally dependent on a number 64 of remaining fractions of a fractionated radiation therapy regimen. If the total number of fractions in the regimen is denoted as N and some number n fractions have been performed thus far, then the remaining number of fractions 64 is equal to N−n. For example, if the current radiation therapy session is the first session then n=0 (the ART recommendation method is performed before actually applying therapeutic radiation in the current session, hence no fractions have yet been performed), then the number of remaining fractions 64 is N. In an operation 66, a decision is made as to whether ART should be performed.

In one approach, the operation 66 includes displaying, on the display 51, 52, 53 of the console 50, a recommendation as to whether adaptive radiotherapy should be performed based on the adaptive radiotherapy recommendation score computed in the operation 62, and receiving, via the at least one user input device 54, 55, a decision as to whether to perform adaptive radiotherapy. If the decision is to not perform adaptive radiotherapy, then the console 50 controls the radiation therapy delivery device 36 to deliver therapeutic radiation to the patient in accord with the radiation therapy plan 20. In this case, the TPS 10 is never involved, and the delay to address the question of whether to perform ART is only a few seconds to a few minutes.

On the other hand, if the decision is to perform adaptive radiotherapy, then the current image 44 is to the TPS 10 which performs the adaptation. The console 50 then receives from the TPS 10 the adapted update of the radiation therapy plan and proceeds to control the radiation therapy delivery device 36 to deliver therapeutic radiation to the patient in accord with the adapted update of the radiation therapy plan. The delay here may be substantially longer, e.g. perhaps an hour or more, in order for the TPS to perform the plan adaptation.

In practice, the PSPM 30 typically models a large number of foreseeable perturbations. For example, it may model growth/shrinkage of the tumor in three dimensions, growth/shrinkage of the urinary bladder in three dimensions, and so forth. In one suitable approach, this is accommodated by the PSPM 30 comprising a plurality of radiation therapy plan-specific perturbation models corresponding to the plurality of different perturbations. Thus, in one implementation covering the foregoing example there is one plan-specific perturbation model for growth/shrinkage of the tumor in the superior-inferior direction, another for growth/shrinkage of the tumor in the posterior-anterior direction, another for growth/shrinkage of the tumor in the lateral direction, another for growth/shrinkage of the urinary bladder in the superior-inferior direction, another for growth/shrinkage of the urinary bladder in the posterior-anterior direction, and another for growth/shrinkage of the urinary bladder in the lateral direction. In one way to combine these, the adaptive radiotherapy recommendation score is computed as the score output by the plurality of radiation therapy plan-specific perturbation models that most strongly indicates that adaptive radiotherapy should be performed. This approach ensures that the ART recommendation is based on the perturbation that has the largest impact. (As an example, if the urinary bladder size is unchanged and the tumor size is unchanged in the superior-inferior and posterior-anterior directions, but the tumor has grown significantly in the lateral direction, then ART should be recommended in order to adjust for the tumor growth in the lateral direction in spite of the lack of change for the other perturbations).

As an additional or variant approach, the operation 66 may display all recommendations based on all computed adaptive radiotherapy recommendation scores output by the plurality of plan-specific perturbation models, with each displayed recommendation being displayed associated with the corresponding perturbation. In the foregoing example, the display could present recommendations for no ART to address urinary bladder size change but also present a recommendation to perform ART to address the lateral growth of the tumor. This approach provides full information to the console operator upon which to make the ART decision. As another contemplated variant, if the console 50 has a “remote app” component, then the display 51, 52, 53 may be the mobile device display of a cellphone or tablet computer of the patient's oncologist or other treating doctor (and likewise the at least one user input device 54, 55 may include at least one input of the cellphone or tablet computer), and the oncologist or other treating doctor then may make the ART decision via the remote app after reviewing the recommendations displayed on the mobile device display.

If the operations 60, 62 are fully automated, for example by using automated DIR to define the contours in the current image 44, then in some embodiments the ART decision is performed automatically or semi-automatically. In such an embodiment, the ART decision 66 is implemented as an alarm. In a typical scenario employing this approach, the current image 44 is acquired in due course of positioning the patient on the support 38 for the radiation therapy session. In an automated fashion the operations 60, 62 are performed on the current image 44 without user intervention, e.g. while the patient is being positioned on the support 38. The operation 66 entails automatically deciding whether to recommend ART, for example a recommendation to perform ART may be generated if the adaptive radiotherapy recommendation score output by the applied PSPM 30 for any analyzed perturbation exceeds some threshold for that perturbation (or, more generally, satisfies an ART recommendation criterion). If a recommendation to perform ART is thereby automatically generated, then this recommendation to perform ART is indicated to the user in the form of an alarm displayed on the display 51, 52, 53. On the other hand, if the automatically generated recommendation is to not perform ART, then no such alarm is displayed (or, alternatively, a message is displayed on the display 51, 52, 53 indicating that adaptive radiotherapy is not recommended for this radiation therapy session).

In the foregoing, it will be appreciated that each of the electronic processors may be embodied by a computer, server, desktop computer, notebook computer, or other microprocessor-based electronic processing device. Each non-transitory storage medium 14, 24, 56 may be variously embodied, e.g. as a hard disk drive, RAID array, or other magnetic storage medium, a solid state drive (SSD) or other electronic storage medium, an optical disk or other optical storage medium, various combinations thereof, and/or so forth. Further, it will be appreciated that the disclosed electronic processors may be variously combined, and/or the various non-transitory storage media 14, 24, 56 may be variously combined. For example, a single server data storage may store the executable code for the TPS 10 and the radiation treatment plans database 22. As noted, the TPS 10 is generally separate from the linac console 50 at least insofar as they are in physically separate locations (e.g. different rooms or hospital floors/suites), employ different user interface devices, and are logically separated (e.g., employing different security passwords or otherwise different user authentication, although it may be that a particular user may be authorized to use both systems 10, 50).

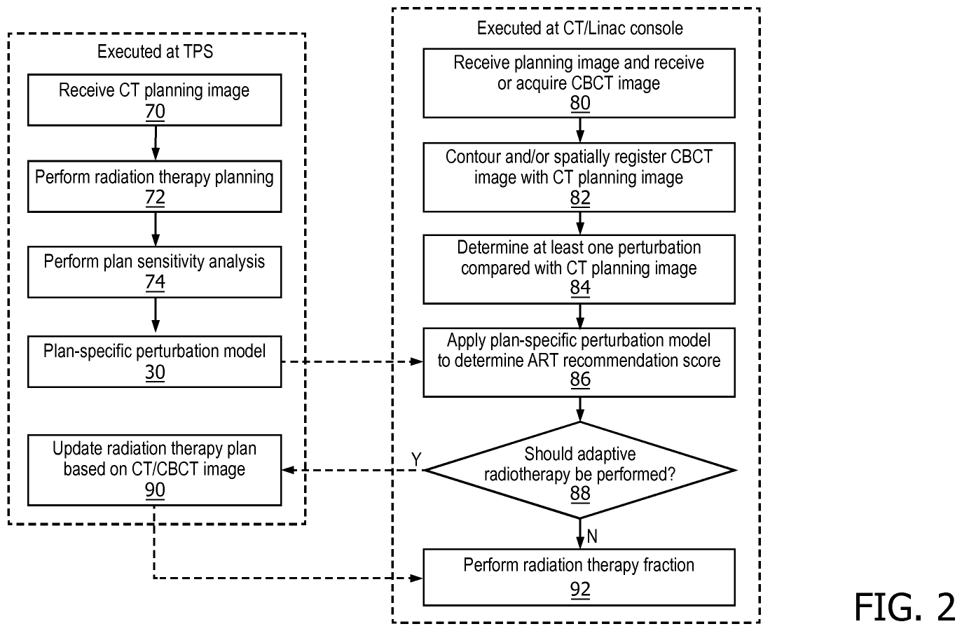

With reference to FIG. 2, an illustrative ART recommendation process performed by the setup of FIG. 1 is illustrated by way of flow charting. In an operation 70, the radiation treatment planning image 1 is received at the TPS 10. In an operation 72, the radiation therapy planning is performed at the TPS 10 to generate the radiation treatment plan 20. As previously mentioned, the operation 72 entails fluence and/or dose distribution simulation for a chosen set of parameter of the radiation therapy delivery device 36 that is destined to deliver the therapeutic radiation, and dose optimization entailing adjusting the parameters to optimize the simulated dose distribution respective to a composite objective function comprising a set of objectives for dosing of the target tumor(s) and OAR(s). Further, while indicated in FIG. 2 as being executed at the TPS, it will be appreciated that the operation 72 may entail some outside consultation, e.g. with the oncologist, and the final radiation treatment plan 20 usually must be approved by the oncologist or other treating physician. In an operation 74, the plan sensitivity analysis (PSA) is performed at the TPS 10 to quantitatively assess the impact of various foreseeable perturbations (e.g. tumor growth or shrinkage, urinary bladder expansion/contraction, et cetera) on a quantification of plan quality (e.g. the value of the composite objective function for the dose distribution for the perturbed anatomy). The output of the PSA 72 is the plan-specific perturbation model (PSPM) 30 which is specific to the radiation treatment plan 20.

The foregoing operations 70, 72, 74 can be viewed as being performed “offline”, that is, prior to commencement of the first fraction of the radiation therapy, in order to generate the treatment plan 20 and (for the ART recommendation process) the PSPM 30. Subsequent ART recommendation operations are performed at the CT/Linac console 50 as discussed next.

In an operation 80, the radiation therapy planning image 1 is retrieved from the database 22 to the linac console 50, and the current image 44 (a CBCT image 44 in the illustrative example) is received or acquired by control (by console 50) of the imaging device 40, 42. In an operation 82 performed at the console 50, features of the current (CBCT) image 44 are contoured at the linac console 50, and the current (CBCT) image 44 and planning image 1 are spatially registered, e.g. by deformable image registration (DIR). The contouring may be performed manually via a graphical user interface (GUI) provided by the console 50, or automatically by energy minimizing contour fitting or the like (preferably with review/adjustment/approval by the console operator). In an operation 84 performed at the console 50, at least one perturbation is determined by comparing the current (CBCT) image 44 with the (spatially registered) planning image 1. This may entail comparison of contours drawn in the current (CBCT) image 44 with corresponding contours drawn of the planning image 1. For example, a perturbation may be identified as a difference (optionally greater than some threshold) between the tumor contour in the current image 44 compared with the tumor contour in the planning image 1. In an operation 86 performed at the console 50, for each identified perturbation the PSPM 30 (retrieved from the database 22 to the console 50) is applied to determine an ART recommendation score for that perturbation. This is a fast and computationally efficient operation, and may in some embodiments entail looking up (and possibly interpolating or extrapolating) the ART recommendation score from a tabulation or look-up table of ART recommendation scores for various perturbation values comprising the PSPM 30. In an operation 88, the ART recommendation decision is made at the linac console 50. This may entail displaying, on the display 51, 52, 53 of the console 50, the ART recommendation scores for each perturbation determined in operation 84 and scored in operation 86. This approach provides the console operator with maximum information upon which to make the decision whether to perform ART. In another approach, the ART recommendation score most strongly indicating that ART should be performed is selected and displayed. This provides less information to the console operator but the information is more concise and should be the most relevant information (e.g. if one perturbation justifies ART while many others do not justify ART, it follows that ART should likely be performed). In yet another embodiment, the decision to perform ART could be fully automated based on the ART recommendation score most strongly indicating that ART should be performed, i.e. if this score is above some threshold for performing ART then the recommendation is to perform ART.

If the decision 88 is that ART should be performed, then the current image 44 is sent to the TPS 10 along with a request to perform ART, and the adaptive radiotherapy optimization is performed in an operation 90 at the TPS 10. This is a computationally complex process involving simulation of dose distribution for the current image 44 and dose optimization by adjusting parameters to optimize the simulated dose distribution respective to the composite objective function. The update adapted radiation treatment plan is then sent back to the linac console 50, and in an operation 92 the radiation therapy (fraction) is performed under control of the linac console 50 in accord with the update adapted radiation treatment plan. On the other hand, if the decision 88 is that ART should not be performed, then the TPS 10 is not consulted (that is, the ART update operation 90 is skipped), and instead process flow transitions directly to the operation 92 at which the radiation therapy (fraction) is performed under control of the linac console 50 in accord with the (original) radiation treatment plan 20.

In the following, some further examples and variant embodiments are described.

After generating a radiation treatment plan 20 based on the original planning (e.g. CT) image 1, and before commencing the first fraction of treatment, the PSA is performed in which sensitivity of the clinical goals (e.g., quantitatively expressed as goals or objectives in some embodiments) to different deformation scenarios is simulated in the original CT using the planning system. For instance, the bladder deformation can be simulated by expanding or contracting the contour of bladder and re-computing the dose statistics and Dose-Volume Histogram (DVH) for the bladder contour accordingly. It is to be noted that the re-computation of dose statistics per deformation scenario is computationally efficient and can be done rapidly and hence a large number of deformation scenarios per organ can be simulated in a short timeframe. The sensitivity can be assessed with respect to dosimetric criteria (or dose-volume criteria), and/or with respect to biological plan evaluation criteria (e.g. biological models). Some suitable biological plan evaluation criteria include Tumor Control Probability (TCP) for tumors and Normal Tissue Complication Probability (NTCP) for normal organs/tissues. As one non-limiting illustrative example, sensitivity to a perturbation can be assessed using TCP, NTCP, and one or more dose volume parameters. The number of such expansion and contraction scenarios and the maximum level of expansion and contraction scenarios are pre-defined in some embodiments. Similarly for all other organs and target volume the same process can be repeated.

As one example, assume that there are N total number of fractions and n fractions have been delivered so far. So N−n fractions are yet to be delivered. Also the total prescribed dose for target is D and dose per fraction is d and hence the tumor is yet to receive a dose of D−nd. The current image 44 is acquired at the (N−n)^{th} fraction to decide of ART is required or not. Considering a prostate tumor case and a bladder expansion scenario at (N−n)^{th} fraction. The prescribed mean dose for Bladder is Dₚᵣₑₛ considering N fractions. If the bladder expands in the anterior-posterior direction, it will get more dose in the remaining fractions. Let us assume that the bladder expansion causes the cumulative mean dose (D_{cum}) to exceed the prescribed mean dose. This is mathematically expressed by:

D_{cum}=Dₘₑₐₙ(n)ΘDₘₑₐₙ(N−n)

where Dₘₑₐₙ(n) is the mean dose to bladder as a result of n delivered fractions and Dₘₑₐₙ(N−n) is the mean dose to bladder as a result of delivering the remaining fractions. Here the symbol Θ denotes the cumulative function over Dₘₑₐₙ(n) and Dₘₑₐₙ(N−n).

It is to be noted that it is not sufficient to simply sum Dₘₑₐₙ(n) and Dₘₑₐₙ(N−n) to obtain D_{cum} because the volumes of the same organ corresponding to n^{th} fraction and (n+1)^{th} fraction are not the same. This calculation can be simulated using TPS 10.

Hence the sensitivity of the bladder to the deformation scenario is computed as below:

Sensitivity=sqrt[(D_{cum}−Dₚᵣₑₛ)²]

Similar such calculations can be performed for other normal organs as well as tumor volume in the plan. The sum of sensitivity of each organ and tumor volume will represent the total impact on the plan due to a combination of deformations.

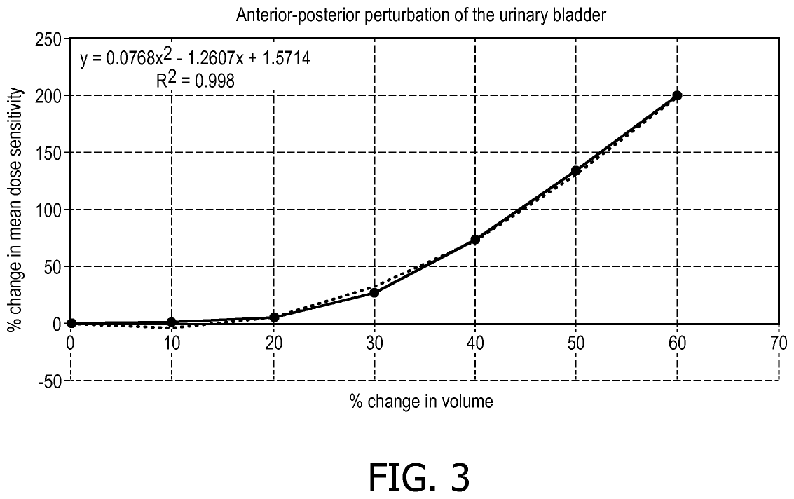

With reference now to FIG. 3, based on the sensitivity analysis, a smooth perturbation model is generated. An example of such a perturbation model is shown in FIG. 3 for the urinary bladder. In the example of FIG. 3, a bladder expansion in the anterior-posterior direction for a prostate case is presented. In FIG. 3, the solid line denotes the percentage change in the mean dose sensitivity with respect to the percentage change in the volume and the dotted line is the fitted curve for the same. The equation displayed inside the graph of FIG. 3 is the model representing how the sensitivity is impacted on introducing perturbations in the bladder contour in anterior-posterior direction. In other words, this equation denotes the clinical objective-specific perturbation with respect to the change in the volume of the corresponding organ applicable for the given patient and plan. In this equation, x denotes the change in volume (along with the direction of change) and y denotes the corresponding change in mean dose sensitivity to the deformations. FIG. 3 plots this for a single radiation treatment session, that is, assuming a fixed number of remaining fractions N−n.

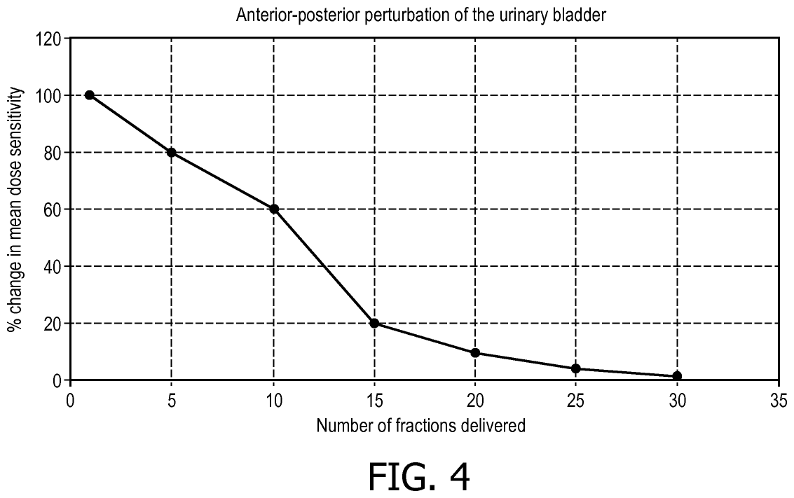

With reference to FIG. 4, a model graph is shown illustrating the percentage change in the mean dose sensitivity with respect to the number of fractions to be delivered out of 30 fractions. FIG. 4 plots this for a certain fixed percentage change in volume of the urinary bladder along the anterior-posterior direction for the prostate case.

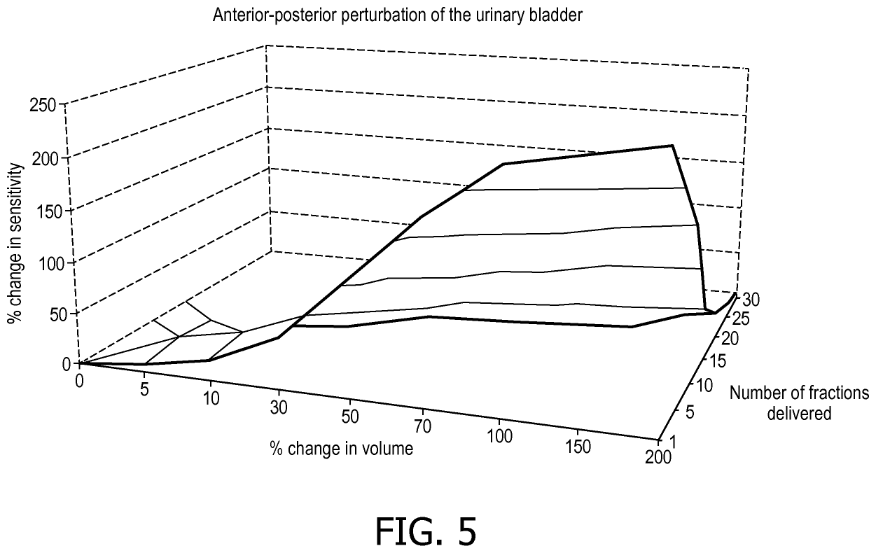

With reference to FIG. 5, foreseeable changes in volume of the urinary bladder along the anterior-posterior direction for the prostate case can be modelled as a PSPM that is functionally dependent on the magnitude of the perturbation (i.e. % change along the anterior-posterior direction) and the number of remaining fractions N−n. This PSPM is depicted in FIG. 5 as a surface plot illustrating an example graph for how the sensitivity is predicted to change with respect to both % change in volume and remaining number of fractions to be delivered out of 30 fractions.

In one illustrative approach, the ART recommendation score is obtained by normalizing the sensitivity from a minimum of 0 to a maximum of 100, and is presented in distinct groups, e.g. such as five groups: 0-10, 11-20, 21-40, 41-70 and 71-100. In this example, the ART recommendation score may be viewed as a risk score, i.e. quantifying the risk of degraded efficacy of the radiation therapy (such that a higher risk constitutes a stronger recommendation to perform ART). The acceptable risk score in this example is obtained from the clinician per anatomic site. A sample template for the perturbation of growth/shrinkage of a prostate tumor in the anterior-posterior direction is shown in Table 1, which tabulates ART decisions for various clinical objectives as a function of ART recommendation score. In this approach, the suggested decision for each clinical objective based on the computed risk score is presented. In general, the higher is the risk score, the higher is the need to adapt the plan. The risk score can be computed for the current patient anatomy obtained from the current image 44 directly at the CT/Linac console 50. The impact on each clinical objective is quickly computed based on the clinical objective-specific perturbation model (PSPM) 30.

The disclosed ART recommendation approaches help the console operator to make meaningful clinical decisions as to whether to perform ART in a quick time (may be a few minutes post image acquisition), and bypasses the need to consult the TPS 10 in deciding the need for ART. This is expected to be very helpful in busy radiation therapy clinics. The disclosed ART recommendation approaches leverage the Linac console 50 to reduce the load on the TPS 10. The load on the TPS 10 respecting the ART recommendation process is reduced to re-computation of dose statistics per deformation scenario during PSA. This is computationally efficient and can be done rapidly; hence, a large number of deformation scenarios per organ can be simulated in a short timeframe. The disclosed ART recommendation approaches also provide a principled and systematic approach for deciding whether to perform ART in a given instance, and significantly reduce patient wait time on the couch. The ART recommendation score accounts for the magnitude of change in anatomy and also the direction of change, hence making the calculations clinically meaningful.

TABLE 1

| | ART recommendation score | | | | |
|---|---|---|---|---|---|
| | 0-10 | 11-20 | 21-40 | 41-70 | 71-100 |
| Tumor-Min Dose | No ART | ART | ART | ART | ART |
| Tumor-Max Dose | No ART | ART | ART | ART | ART |
| Rectum-Mean Dose | No ART | No ART | ART | ART | ART |
| Rectum-Max dose | No ART | No ART | ART | ART | ART |
| Bladder-Mean Dose | No ART | No ART | No ART | ART | ART |
| Bladder-Max dose | No ART | No ART | No ART | ART | ART |
| Femur-max dose | No ART | No ART | No ART | No ART | ART |

Since the PSA and PSPM calculations explicitly account for the remaining fractions to be delivered, the resulting risk score will not be overly sensitive to the anatomic deformations. This approach allows the risk score to be indicative of the actual clinical situation at a given fraction and hence leading to optimal clinical decisions. Significantly, by taking into account the number of remaining fractions N−n, the likelihood of recommending ART is reduced for the later fractions, as illustrated in FIGS. 4 and 5. Conceptually, this captures the insight that if the anatomy changes near the end of the fractionated radiation therapy regimen then the total amount of “incorrect” dosage to the tumor and/or OAR is reduced since most of the total dosage has already been delivered, and so the benefit of ART is effectively reduced. By contrast, if the anatomy changes near the beginning of the fractionated radiation therapy regimen then the total amount of “incorrect” dosage to the tumor and/or OAR is high since most of the total dosage is yet to be delivered in future fractions, making ART more beneficial.

By contrast, when using the conventional approach, the current image is sent to the TPS and the decision on whether to perform ART is made at the TPS. In this approach, the radiation physicist may have a tendency to consider only the extent to which the current image deviates from (i.e. is perturbed respective to) the original planning image, and may be less likely to take into consideration the number of remaining fractions.

The invention has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A non-transitory storage medium storing instructions readable and executable by a console including a display, at least one user input device, and an electronic processor to perform a method comprising:
determining at least one perturbation of a current image compared with a radiation therapy planning image used to generate a radiation therapy plan;
computing an adaptive radiotherapy recommendation score indicating whether adaptive radiotherapy should be performed by operations including applying a radiation therapy plan-specific perturbation model that is specific to the radiation therapy plan and is functionally dependent on the determined at least one perturbation; and
displaying, on the display of the console, one of (i) a recommendation as to whether adaptive radiotherapy should be performed based on the computed adaptive radiotherapy recommendation score and (ii) an alarm conditional upon the computed adaptive radiotherapy recommendation score satisfying an ART recommendation criterion.

2. The non-transitory storage medium of claim 1 wherein the radiation therapy plan-specific perturbation model is further functionally dependent on a number of remaining fractions of a fractionated radiation therapy regimen.

3. The non-transitory storage medium of claim 1 wherein:
the at least one perturbation comprises a plurality of different perturbations, and
the radiation therapy plan-specific perturbation model comprises a plurality of radiation therapy plan-specific perturbation models corresponding to the plurality of different perturbations, and
the adaptive radiotherapy recommendation score is computed as the score output by the plurality of radiation therapy plan-specific perturbation models that most strongly indicates that adaptive radiotherapy should be performed.

4. The non-transitory storage medium of claim 1 wherein:
the at least one perturbation comprises a plurality of different perturbations, and
the radiation therapy plan-specific perturbation model comprises a plurality of radiation therapy plan-specific perturbation models corresponding to the plurality of different perturbations, and
the displaying comprises displaying a plurality of recommendations as to whether adaptive radiotherapy should be performed based on the computed adaptive radiotherapy recommendation scores output by the plurality of radiation therapy plan-specific perturbation models, with each displayed recommendation being displayed associated with the corresponding perturbation.

5. The non-transitory storage medium of claim 1 wherein the determining and the computing are performed automatically without being based on input received via the at least one user input device (54, 55) and the displaying comprises displaying said alarm conditional upon the computed adaptive radiotherapy recommendation score satisfying said ART recommendation criterion.

6. The non-transitory storage medium of claim 1 wherein the at least one perturbation includes a combined perturbation comprising two or more individual perturbations.

7. The non-transitory storage medium of claim 1 wherein the determining of at least one perturbation of the current image compared with the radiation therapy planning image includes:
spatially registering the current image and the radiation therapy planning image;
contouring at least one feature in the current image; and
determining said at least one perturbation as a change in the at least one feature contoured in the current image compared with the at least one feature contoured in the spatially registered radiation therapy planning image.

8. The non-transitory storage medium of claim 1 wherein the computing of the adaptive radiotherapy recommendation score indicating whether adaptive radiotherapy should be performed does not include simulating a dose distribution in a patient as represented by the current image.

9. The non-transitory storage medium of claim 8 wherein the method further includes:
receiving, via the at least one user input device of the console, an indication to not perform adaptive radiotherapy; and
subsequent to receiving the indication to not perform adaptive radiotherapy, operating a radiation therapy delivery device operatively connected with the console to deliver therapeutic radiation to a patient in accord with the radiation therapy plan.

10. The non-transitory storage medium of claim 8 wherein the method further includes:
receiving, via the at least one user input device of the console, an indication to perform adaptive radiotherapy and in response transmitting the current image to a Treatment Planning System (TPS) and receiving from the TPS an adapted update of the radiation therapy plan; and
operating a radiation therapy delivery device operatively connected with the console to deliver therapeutic radiation to a patient in accord with the adapted update of the radiation therapy plan;
wherein the method does not include performing adaptive radiotherapy.

11. A console comprising:
a display;
at least one user input device;
an electronic processor; and
a non-transitory storage medium storing instructions readable and executable by the electronic processor to control a radiation therapy delivery device operatively connected with the console and to perform a method including:
receiving a current image of a patient;
determining at least one perturbation of the current image compared with a radiation therapy planning image from which a radiation therapy plan for the patient has been generated;
computing an adaptive radiotherapy recommendation score indicating whether adaptive radiotherapy should be performed based on the determined at least one perturbation; and
displaying, on the display, one of (i) a recommendation as to whether adaptive radiotherapy should be performed based on the computed adaptive radiotherapy recommendation score and (ii) an alarm conditional upon the computed adaptive radiotherapy recommendation score satisfying an ART recommendation criterion.

12. The console of claim 11 wherein the adaptive radiotherapy recommendation score is computed by operations including applying a radiation therapy plan-specific perturbation model that is specific to the radiation therapy plan for the patient and is functionally dependent on the determined at least one perturbation.

13. The console of claim 12 wherein the radiation therapy plan-specific perturbation model (30) is further functionally dependent on a number of remaining fractions of a fractionated radiation therapy regimen of the patient.

14. The console of claim 11 wherein:
the at least one perturbation comprises a plurality of different perturbations, and
the computing of the adaptive radiotherapy recommendation score includes computing an adaptive radiotherapy recommendation score for each perturbation of the plurality of different perturbations.

15. The console of claim 11 wherein the determining of at least one perturbation of the current image compared with the radiation therapy planning image includes:
spatially registering the current image and the radiation therapy planning image;
contouring at least one feature in the current image; and
determining said at least one perturbation as a change in the at least one feature contoured in the current image compared with the at least one feature contoured in the spatially registered radiation therapy planning image.

16. The console of claim 11 wherein the method further includes, after the displaying:
receiving, via the at least one user input device, a decision as to whether to perform adaptive radiotherapy;
conditional upon the decision being to not perform adaptive radiotherapy, controlling the radiation therapy delivery device to deliver therapeutic radiation to the patient in accord with the radiation therapy plan; and
conditional upon the decision being to perform adaptive radiotherapy, transmitting the current image to a Treatment Planning System (TPS) and receiving from the TPS an adapted update of the radiation therapy plan and controlling the radiation therapy delivery device to deliver therapeutic radiation to the patient in accord with the adapted update of the radiation therapy plan.

17. The console of claim 11 wherein the display comprises a mobile device display of a cellphone or tablet computer and the at least one user input device comprises at least one input of the cellphone or tablet computer.

18. A radiation therapy delivery system comprising:
a radiation therapy delivery device configured to deliver therapeutic radiation to a patient disposed on a patient support;
an imaging device configured to image the patient disposed on the patient support of the radiation therapy delivery device; and
a console as set forth in claim 17 operatively connected to control the radiation therapy delivery device and to control the imaging device.

19. The console or radiation therapy delivery system of claim 11 wherein the radiation therapy delivery device comprises a linear accelerator (linac) and the imaging device comprises a computed tomography (CT) scanner.

20. An adaptive radiotherapy recommendation method comprising:
determining at least one perturbation of a current image of a patient compared with a radiation therapy planning image of the patient from which a radiation therapy plan for the patient has been generated;
computing an adaptive radiotherapy recommendation score indicating whether adaptive radiotherapy should be performed based on the determined at least one perturbation and without simulating a dose distribution in the patient as represented by the current image; and
controlling a display to present one of (i) a recommendation as to whether adaptive radiotherapy should be performed based on the computed adaptive radiotherapy recommendation score and (ii) an alarm conditional upon the computed adaptive radiotherapy recommendation score satisfying an ART recommendation criterion;
wherein the adaptive radiotherapy recommendation method is performed by an electronic processor.

21. The adaptive radiotherapy recommendation method of claim 20 wherein the adaptive radiotherapy recommendation score is computed by operations including applying a radiation therapy plan-specific perturbation model that is specific to the radiation therapy plan for the patient and is functionally dependent on the determined at least one perturbation.

22. The adaptive radiotherapy recommendation method of claim 20 further comprising:
receiving a decision via a user input device as to whether to perform adaptive radiotherapy in response to the presentation of the recommendation; and
responsive to the decision being to perform adaptive radiotherapy, transmitting the current image to a Treatment Planning System (TPS) and receiving from the TPS an adapted update of the radiation therapy plan;
wherein the processor that performs the adaptive radiotherapy recommendation method is not a component of the TPS.
